(19) United States
(12) Patent Application Publication
(10) Pub. No.: US 2021/0378639 A1
(43) Pub. Date: Dec. 9, 2021

(54) Method for detecting antigens or antibodies to the antigens using EBC and EBC collection device

(71) Applicants: T. Que COLLINS, Los Angeles, CA (US); Christopher Brian REID, Los Angeles, CA (US)

(72) Inventors: T. Que COLLINS, Los Angeles, CA (US); Christopher Brian REID, Los Angeles, CA (US)

(21) Appl. No.: 17/338,736

(22) Filed: Jun. 4, 2021

Related U.S. Application Data
(60) Provisional application No. 63/034,409, filed on Jun. 4, 2020.

(51) Int. Cl. A61B 10/00; G01N 33/543

(52) U.S. Cl. CPC A61B 10/00; A61B 2010/0087; G01N 33/54386

(57) ABSTRACT

An exhaled breath condensate (EBC) collection device comprising two adjacent parts foldable together and apart from each other, a condensation zone to condense exhaled breath from a subject, the condensation zone being a third part provided between the two adjacent parts and connectable to cooling means arranged in each of the two parts being in cooling relationship with the condensation zone. There is also provided a method for detecting antigens or antibodies to the antigens, the method comprising collecting EBC from a subject using the EBC collection device, performing immunochromatographic testing (ICT) of the collected exhaled breath condensate, and interpreting the immunochromatographic testing results as follows: if the immunochromatographic testing results are positive, the antigens or antibodies to the antigens are detected, or if the immunochromatographic testing results are negative, the antigens or antibodies to the antigens are not detected.

### CROSS-REFERENCE TO RELATED APPLICATIONS

This application claims the benefit of U.S. Provisional Patent Application No. 63/034,409 filed on Jun. 4, 2020, which is incorporated by reference herein in its entirety.

### BACKGROUND OF THE INVENTION

### 1. Field of the Invention

The present invention relates to a method for detecting antigens or antibodies to the antigens, and an exhaled breath condensate (EBC) collection device used in the method.

### 2. Discussion of Background Information

Human respiratory tract infections represent the most commonly encountered infections worldwide.

Challenges with Diagnosis

COVID-19 affects different people in different ways. Infected people have had a wide range of symptoms reported—from mild symptoms to severe illness.

Treatment There is no specific antiviral treatment recommended for COVID-19.

Prevention

Currently, the best way to prevent illness and reduce transmission is to prevent individuals from being exposed to the virus. Accordingly, identification of individuals infected with the virus, including those infected asymptomatically, is critical to individual protection and public health efforts.

Exhaled Breath Condensates
In 2019, Amor et al. wrote, “A highly promising development in cancer biomarkers is based on volatolomics, i.e. on analysis of volatile organic compounds (VOCs) (semi-volatile and highly volatile) that emanate from cancer cells and/or their micro-environment and that are detectable in different body fluids (exhaled breath, blood, urine, sweat, etc.) depending on their tissue/blood and blood/air partition coefficients [28]. The rationale behind this approach rests on the fact that, during pathophysiological processes, alterations in cell metabolism lead to shifts in the production of VOCs, which are by-products of biochemical reactions [29]. Hypoxia, hyperproliferation of cells, excessive inflammatory and reactive oxygen species activity, and other cancer-related pathological mechanisms result in marked shifts in the spectra and concentrations of VOCs, both locally and systematically [30].”

Exhaled Breath Condensates (EBCs) “mainly consist of water vapour but a small fraction contains respiratory droplets derived from the airway lining fluid . . . . This observation has created a growing interest in the use of EBC as a new sampling method for the screening of respiratory viruses infecting the upper airways” (Houspie et al. 2011).

More recently, Khoubnasabjafari et al. (2020) have speculated that Exhaled breath condensate may be feasible for diagnosing COVID-19. They write: “We propose that EBC samples should be tested as a non-invasive sampling method in clinics, since it seems a promising specimen for diagnosis of patients with COVID-19 infections. EBC sampling could be performed as many times as needed in follow-up investigations of patients, which is not possible with BLF sampling. Unfortunately, our team does not currently have access to a safety level 3 laboratory in our research center to test the applicability of this hypothesis. There is also a possibility of developing microfluidics or other single-use technologies to collect and analyze the sample.”

In contrast to Khoubnasabjafari et al., the current invention, in part, comprises a method of testing for COVID-19 that can be taken at home or any other setting.

Breath Condensers

EBC can be collected using a variety of breath condensers featuring various manner of construction.

Krishnan and Warwick write, “collection devices significantly affect exhaled breath condensate biomarker levels and absolute values from different devices are not directly comparable. Siliconised glass showed the least variability in oxides of nitrogen levels, whilst the EcoScreen was more consistent and efficient for protein collection, and also in terms of total exhaled breath condensate volume. This efficiency may in part be related to the lower temperature used in this device. We conclude that no single device is ideal for all applications. In future studies, each marker may need to be tested in a variety of devices to determine the optimal collection apparatus.”

Testing

Collected EBC can be analyzed using genetic and serologic testing according to various published protocols suitable for use in the present invention.

For example, the Johns Hopkins Center for Health Security's statement on PCR Diagnostic Testing for SARS-CoV-2 (Apr. 17, 2020) states,

“Diagnostic testing for the novel coronavirus SARS-CoV-2 is undertaken using 3 approaches: whole genome sequencing, real-time reverse transcriptase PCR (rRT-PCR), and serology . . . . Currently, almost all diagnostic testing for SARS-CoV-2 is done using rRT-PCR.” https://www.centerforhealthsecurity.org/resources/COVID-19/COVID-19-fact sheets/200130-nCoV-diagnostics-factsheet.pdf

A person in the United States can be tested for SARS-CoV-2 by a physician's order when that person has symptoms consistent with COVID-19, the disease caused by the SARS-CoV-2 virus; however, more widespread, reliable testing is desirable, including at home testing. The current invention represents a simple, cost-effective means of increasing testing access.

“Testing is of high priority in

1. Hospitalized patients and healthcare facility workers,

2. Patients in at-risk populations: patients in long-term care facilities, patients over the age of 65, patients with comorbidities (diabetes, cardiovascular disease, chronic lung disease), and first responders with symptoms.” https://www.centerforhealthsecurity.org/resources/COVID-19/COVID-19-fact-sheets/200130-nCoV-diagnostics-factsheet.pdf

PCR Testing

“Protocols for rRT-PCR testing developed by several countries and entities, including Germany, Hong Kong, China CDC, Thailand, and Japan, have been posted to the WHO website,2 and the protocol for testing in the United States has been posted to CDC's site.3 Charité Universitatsmedizin Berlin developed the first assay and protocol.

US Protocol: rRT-PCR tests for SARS-CoV-2 infection in the United States are being run at the CDC in Atlanta for jurisdictions that do not have local testing available. The CDC has published a list of diagnostic rRT-PCR tests that have Emergency Use Authorization (EUA) approval.

rRT-PCR primers and probes have been published by CDC.

Materials and reagents required to run the rRT-qPCR tests are listed here.

Commercial, hospital-based, and academic rRT-PCR tests kits are now widely available.”

### REFERENCES

1. US Centers for Disease Control and Prevention. Evaluating and testing persons for coronavirus disease 2019 (COVID-19). Reviewed Mar. 24, 2020. https://www.edc.gov/coronavirus/ SARS-CoV-2/clinical-criteria.html. Accessed Apr. 8, 2020.
2. World Health Organization. Coronavirus disease (COVID-19) technical guidance: laboratory testing for 2019-nCoV in humans. Mar. 31, 2020. https://www.who.int/emergencies/diseases/novel-coronavirus-2019/technical-guidance/laboratory-guidance. Accessed Apr. 8, 2020.
3. US Centers for Disease Control and Prevention. CDC 2019-novel coronavirus (2019-nCoV) real-time RT-PCR diagnostic panel. Mar. 30, 2020. https://www.fda.gov/media/164922/download. Accessed Apr. 8, 2020.
4. US Food and Drug Administration. Emergency Use Authorization: coronavirus disease 2019 (COVID-19) Emergency Use Authorizations for medical devices. Apr. 7, 2020. https://www. fda.gov/medical-devices/emergency-situation-medical-devices/ emergency-use-authorizations#covid19ivd. Accessed Apr. 8, 2020.
5. Zhu N, Zhang D, Wang W, et al. A novel coronavirus from patients with pneumonia in China, 2019. N Eng J Med 2020; 382(8):727-733.
6. Chan J F W, Yuan S, Kok K H, et al. A familial cluster of pneumonia associated with the 2019 novel coronavirus indicating person-to-person transmission: a study of a family cluster. Lancet 2020; 395(10223):514-523.
7. Janus J. Using whole genome sequencing to help combat COVID-19. University of Cambridge. Mar. 27, 2020. https://www.phgfoundation.org/blog/wgs-to-combat-COVID-19. Accessed Apr. 8, 2020.
8. NextStrain. Genomic epidemiology of novel coronavirus—global subsampling. Apr. 5, 2020. https://nextstrain.org/ncov/global. Accessed Apr. 8, 2020.
9. US Centers for Disease Control and Prevention. Interim laboratory biosafety guidelines for handling and processing specimens associated with coronavirus disease 2019 (COVID-19). Reviewed Mar. 31, 2020. https://www.cdc.gov/coronavirus/2019-ncov/lab/ lab-biosafety-guidelines.html. Accessed Apr. 8, 2020.
10. US Food and Drug Administration. Coronavirus disease 2019 (COVID-19). Apr. 7, 2020. https://www.fda.gov/emergency-preparedness-and-response/counterterrorism-and-emerging-threats/coronavirus-disease-2019-covid-19. Accessed Apr. 8, 2020. © Johns Hopkins Center for Health Security, centerfothealthsecurity.org Apr. 17, 2020

Serologic Testing

According to the Johns Hopkins Center on Health Security, “Serology tests are blood-based tests that can be used to identify whether people have been exposed to a particular pathogen.”

https:/www.centerforhealthsecurity.org/resources/COVID-19/COVID-19-fact-sheets/200228-Serology-testing-COVID.pdf

Serology-based tests analyze the serum component of whole blood. The serum includes antibodies and antigens.

“There are Several Types of Serology Tests

1. Neutralization tests can indicate whether the patient has

active, functional antibodies to the pathogen in question by measuring how much the patient antibodies can inhibit viral growth in the lab.

2. Immunofluorescent assay (IFA) shows whether a patient has antibodies to a pathogen by displaying a fluorescent signal when patient antibodies interact with virus proteins.

3. Enzyme-linked immunosorbent assays (ELISAs) are more rapid serology tests that provide a readout of antigen-antibody interactions. Essentially, patient antibodies are “sandwiched” between the viral protein of interest and reporter antibodies, so that any active patient antibodies are detected—(see FIG. 1).

4. Western blot tests, which can be used in laboratory settings to detect the presence of a protein of interest by the emission of a colored or fluorescent reporter when an active antibody interacts with the viral protein.”

All such serology tests are suitable for use in the present invention.

Structural Analysis of COVID-19

Coronavirus has only 4 structural proteins, the spike (S), membrane (M), envelope (E), and nucleocapsid (N) proteins.

Serology testing for COVID-19 is of critical importance because of the relatively short time to diagnosis and the ability to test for an active immune response against the virus.

Having compatible genetic and serological EBC based tests allows for confirmation of infection, rather than relying on a single approach. Likewise, multiple potential serology tests for COVID-19 would provide (1) one or more test(s) for the presence of viral antigens and (2) one or more test(s) that detect the presence of antibodies to the virus.

Immunochromatographic Testing (ICT)

Lateral flow immunochromatographic tests (ICTs), for example, have been primarily developed for rapid field testing, but have also been incorporated in clinical laboratories. These tests usually consist of single-use, disposable cartridges or strips which generate detectable colored end products interpreted as positive or negative.

In view of the foregoing, there is a need for a rapid and efficient analysis for the presence of antigens and/or antibodies, which is less invasive and allows testing at home or in almost any other setting, not only in clinical settings, and which is useful in any disease or clinical condition in which an ultrafiltrate, such EBC, can be assessed.

### SUMMARY OF THE INVENTION

The present invention provides an exhaled breath condensate (EBC) collection device comprising two adjacent parts foldable together and apart from each other, a condensation zone to condense exhaled breath from a subject, the condensation zone being a third part provided between the two adjacent parts and connectable to cooling means arranged in each of the two parts being in cooling relationship with the condensation zone.

In one particular embodiment of the device, the two adjacent parts and the condensation zone are essentially flat.

In another particular embodiment of the device, the cooling means are freezer packs or ice packs.

In another particular embodiment, the device further comprises an inlet port enabling the subject to breathe into the condensation zone through a detachable mouth-piece.

In another particular embodiment of the device, the condensation zone comprises an exhaled breath condensate collection area in a bottom area of the condensation zone, enabling the generated breath condensate flow down to the collected area for subsequent collection for performing tests.

In another particular embodiment, the device further comprises a perforated label connecting the two adjacent parts in a folded-together state.

In another particular embodiment, the device further comprises a breathing indicator that is indicative of a rate of exhaled breath.

The present invention also provides a method for detecting antigens or antibodies to the antigens, the method comprising:
collecting exhaled breath condensate (EBC) from a subject using an exhaled breath condensate collection device comprising two adjacent parts foldable together and apart from each other, a condensation zone to condense exhaled breath from a subject, the condensation zone being a third part provided between the two adjacent parts and connectable to cooling means arranged in each of the two parts being in cooling relationship with the condensation zone,
performing immunochromatographic testing (ICT) of the collected exhaled breath condensate, and
interpreting the immunochromatographic testing results as follows:
   if the immunochromatographic testing results are positive, the antigens or antibodies to the antigens are detected, or
   if the immunochromatographic testing results are negative, the antigens or antibodies to the antigens are not detected.

In one particular embodiment, the antigens are proteins.

In another particular embodiment, the method includes using a smartphone-based camera and/or app to perform quantitative assessments.

In yet another particular embodiment of the method, the collecting of the EBC from the subject includes respiring by the subject through the EBC collection device at least for 5 minutes.

### BRIEF DESCRIPTION OF THE DRAWINGS

The present invention is further described in the detailed description which follows, in reference to the drawings wherein:

FIG. 1 illustrates a chart of different phases of Covid-19 infection.

FIG. 2 illustrates a general view of the EBC collection device.

FIG. 3 illustrates widely used blood-based tests.

FIG. 4 illustrates assembling a mouthpiece to the EBC collection device.

FIG. 5 illustrates the EBC collection device with the parts in a folded-together state and in foldable apart from each other state.

FIG. 6 illustrates the EBC collection device in a freezer.

FIG. 7 illustrates a mouthpiece in a protective plastic bag.

FIG. 8 and FIG. 9 illustrate the EBC collection device and the mouthpiece.

FIG. 10 shows using of the EBC collection device by a subject.

FIG. 11 illustrates removal of the mouthpiece from EBC collection device.

FIG. 12 illustrates how a perforated label on the right-hand side of the device may be broken.

FIG. 13 illustrates the EBC collection device with the parts folded apart from each other.

FIG. 14 illustrates the EBC collection device laid face down with the back part opened.

FIG. 15 illustrates an enlarged view of a small label covering the fluid collection area of the EBC collection device shown on FIG. 14.

FIG. 16 illustrates drawing out the required quantity of fluid (e.g. by pipette) for tests.

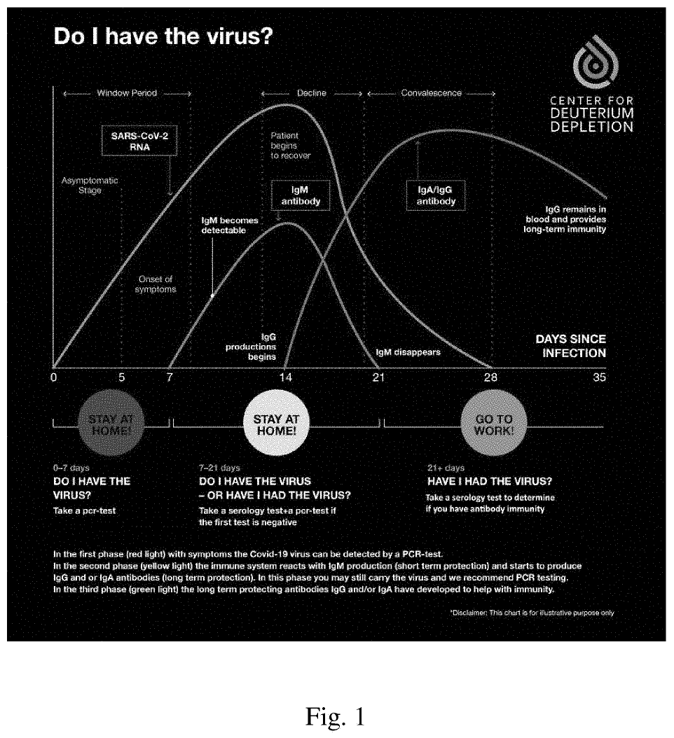

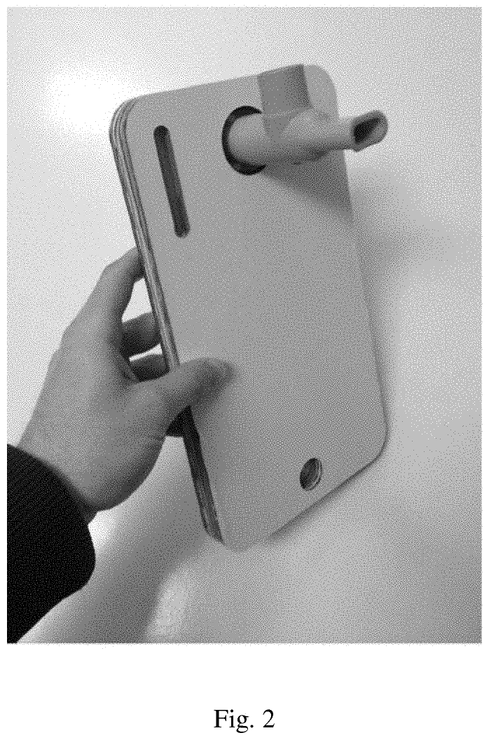

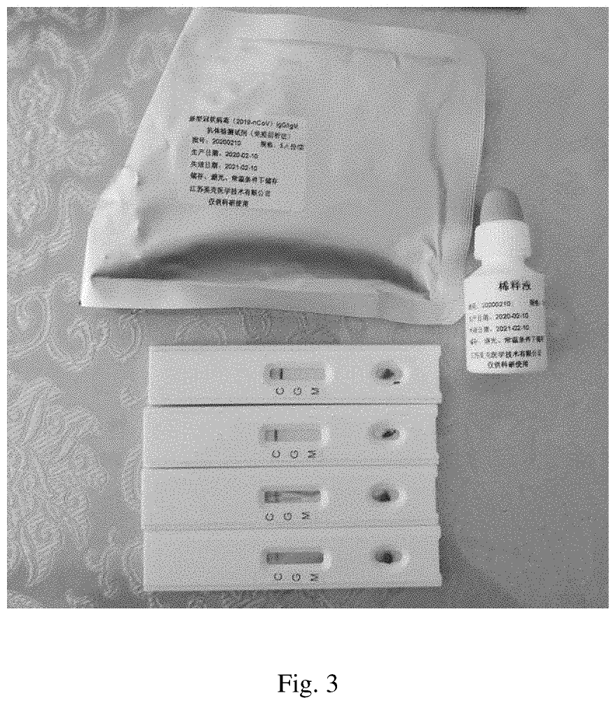

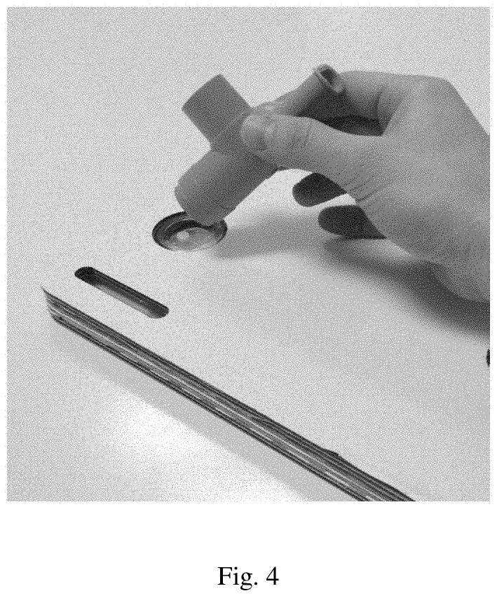

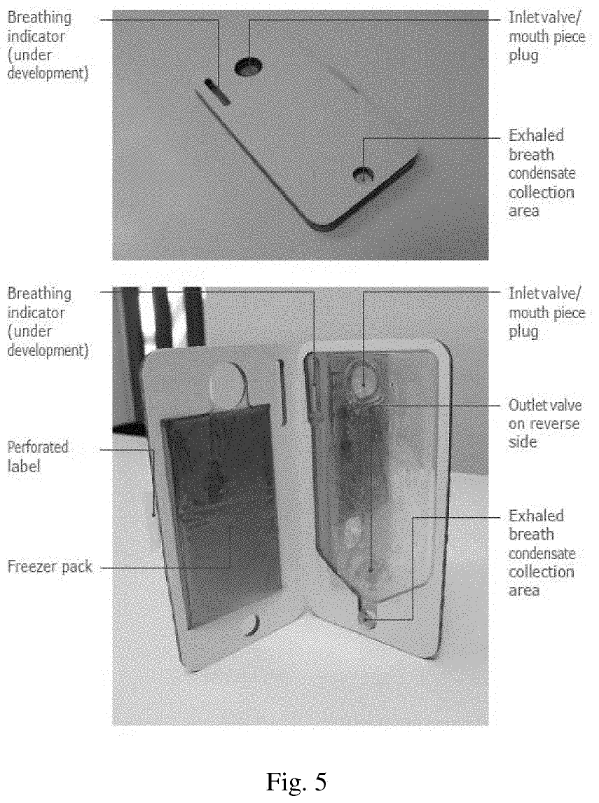

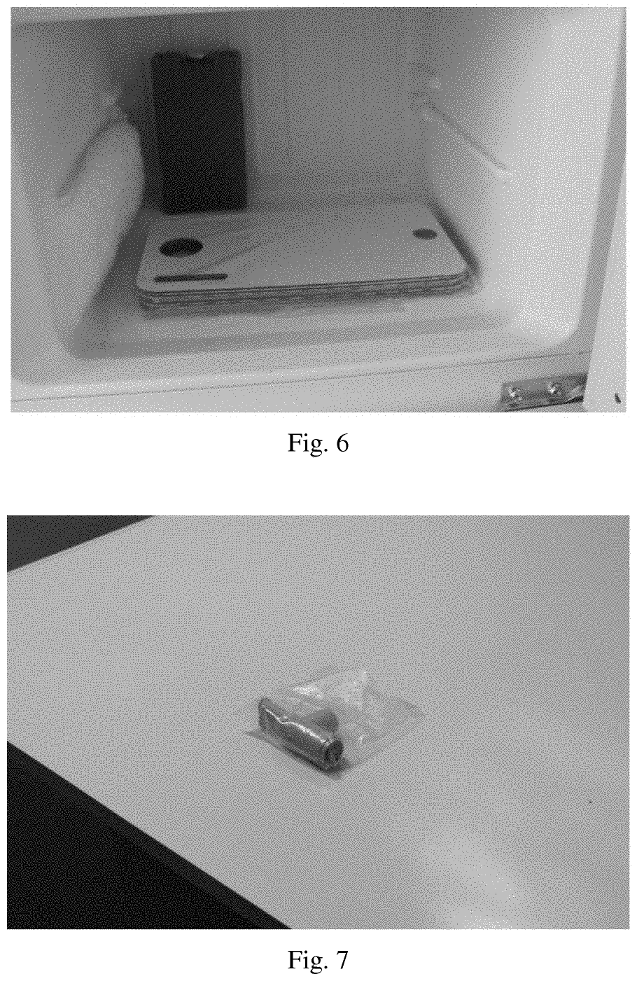

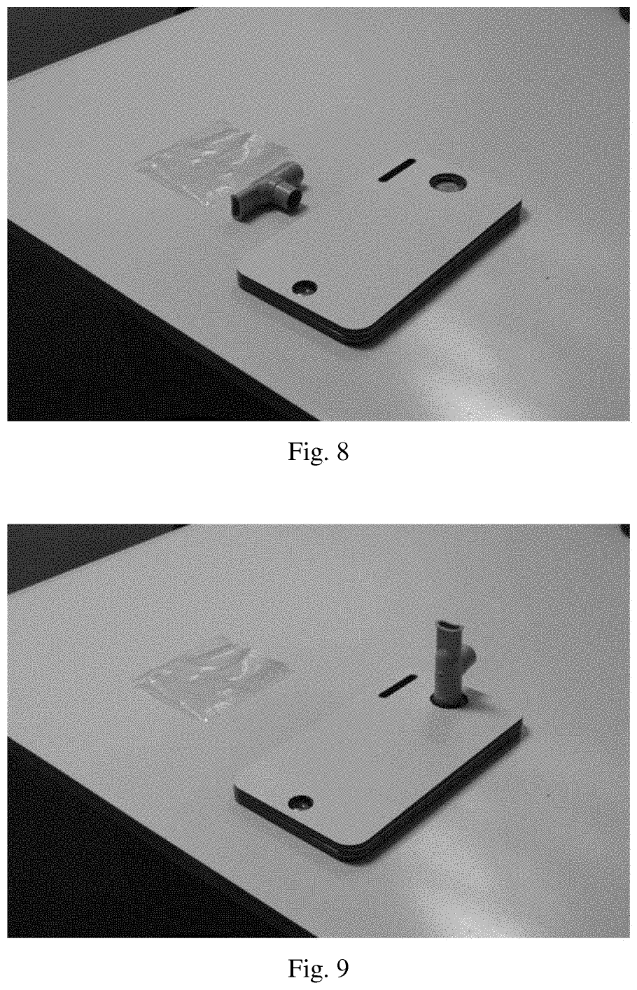

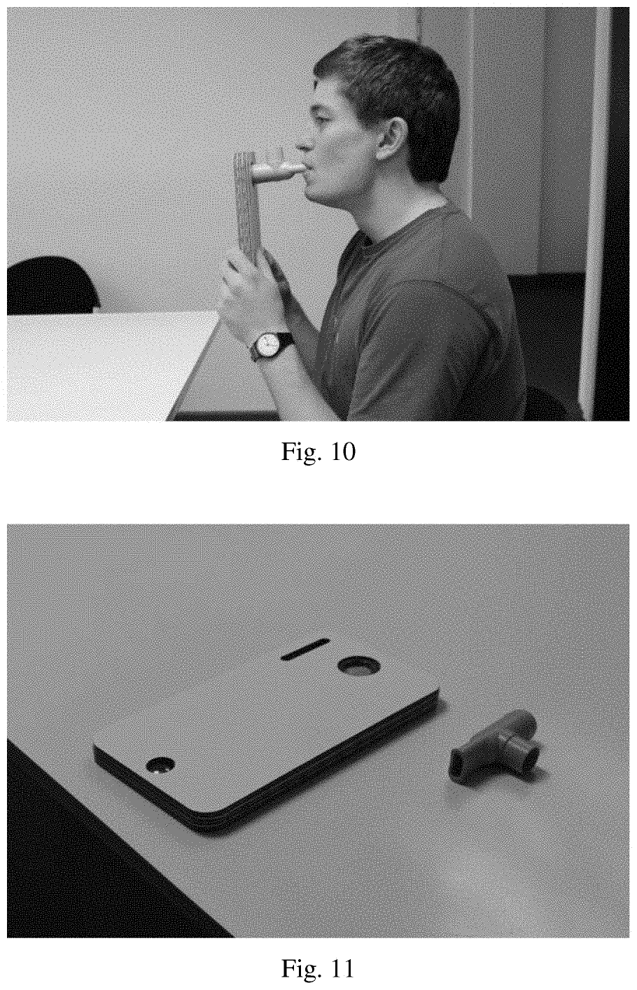

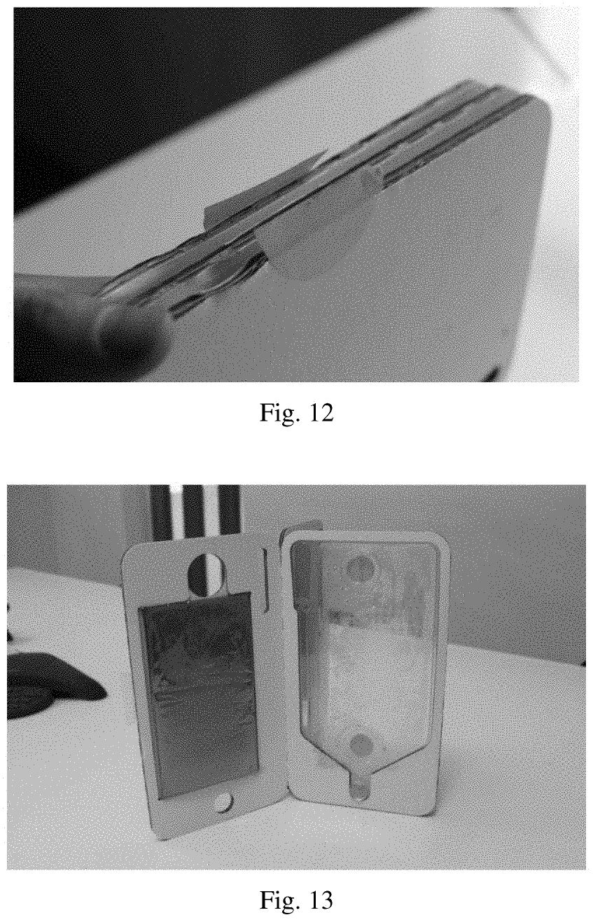

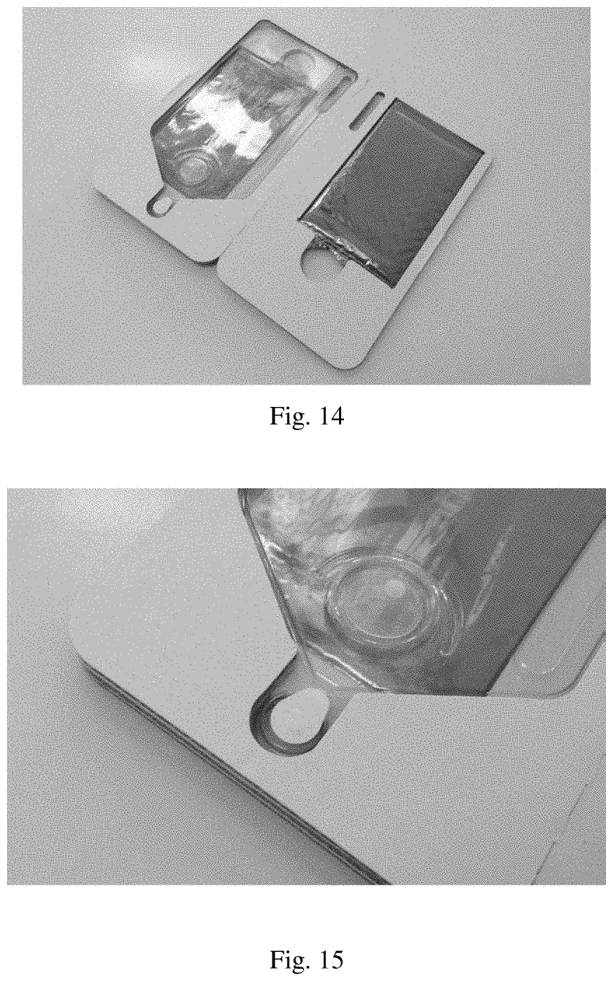

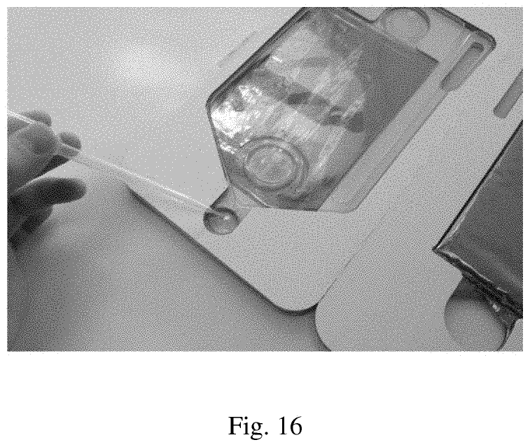

### DETAILED DESCRIPTION OF THE EMBODIMENTS

The subject-matter of the present application is a method for detecting antigens or antibodies to the antigens, the method comprising collecting exhaled breath condensate (EBC) from a subject using an exhaled breath condensate collection device, performing immunochromatographic testing (ICT) of the collected exhaled breath condensate, interpreting the immunochromatographic testing results as follows:
if positive, the antigens or antibodies to the antigens are detected, or
if negative, the antigens or antibodies to the antigens are not detected.

In the present invention, the ICT may be applied to all manner of blood ultrafiltrates for non-invasive genetic testing, and/or serologic testing, including in the context of various clinical conditions and disorders.

ICT testing applied to a blood ultrafiltrate may be aimed at detecting either antigens or antibodies. For example, various cancer antigens, viral antigens, disease related antigens, disease related antibodies, rheumatoid related antibodies, etc. can be detected in this manner. Moreover, immunofluorescent ICT allows for the target antigens and/or antibodies to be quantified. Urusov, et al., (2019) write:

“Quantitative estimation of the results of immunochromatographic tests can substantially enhance the efficiency of their use in human and veterinary medicine, biosafety, consumer protection, and ecological monitoring. Actually, a row of portable detectors has been developed and implemented with the corresponding software for different kinds of immunochromatographic tests, including assays with optical, magnetic, and electrically conductive labels. While retaining the speed, cost effectiveness, and consumer friendliness of the test systems, these detectors enable detecting and processing results while precluding the subjectivity of their interpretations. New solutions based on the use of general-purpose devices such as office scanners, mobile phones, and smartphones are being developed.”

In some embodiments, the present invention includes the use of a smartphone-based camera and/or app to achieve quantitative assessments.

In other embodiments, PCR amplification of viral nucleic acid sequences in the EBC and/or other serological methods detecting antigen or antibody to antigen, as alternative to the ICT, can be also envisaged and covered by the present disclosure.

Accordingly, the present invention enables rapid and efficient detection of various antigens, including proteins, or antibodies to those antigens, using EBC (or, alternatively, other blood ultrafiltrates such as urine and saliva) and does not require blood sampling.

Furthermore, an exemplary and novel exhaled breath condensate (EBC) collection device (licensed to be used in the above method) is described, comprising two adjacent parts, wherein the two parts are foldable together and apart from each other, a condensation zone to condense exhaled breath from a subject, the condensation zone being the third part provided between the two parts and connectable to a cooling means arranged in each of the two parts being in cooling relationship with the condensation zone.

The two foldable parts and the condensation zone part are essentially flat, thereby advantageously making the EBC collection device portable and very compact. The cooling means can be freezer packs or ice packs.

The EBC collection device further comprises an inlet port to enable a user to breathe into the condensation zone of the device through a detachable, mouth-piece. The condensation zone comprises an exhaled breath condensate collection area in the bottom area of the condensation zone, thereby enabling the generated breath condensate flow down to the collected area for subsequent collection (e.g. by pipette) for performing tests.

The EBC collection device also comprises a perforated label connecting two parts in a folded-together state.

The EBC collection device may further comprise a breathing indicator that is indicative of a rate of exhaled breath.

Operation of the EBC Collection Device:

The EBC collection device is kept inside the protective plastic bag and placed in a freezer overnight. The mouthpiece is prepared to be in an easily accessible location ready to be used during the tests.

The EBC collection device is removed from the freezer and is taken out from its protective plastic bag. A mouthpiece is also removed from its protective plastic bag, inserted into the inlet port of the EBC collection device. The inlet port is positioned centrally at the top of the EBC collection device with the breathing indicator to its left.

The ready-to-use EBC collection device is handed to a subject. The subject respires through the EBC collection device for about 10 minutes, or for 5 minutes at least. If at any point the subject needs to stop to catch his/her breath, the timer should be paused and then recommenced when he/she is ready to do so for the remaining duration of the test. It is important that the subject respires through a nose during inhalation and through a mouth during exhalation. Subject's condition permitting, he/she can be instructed to perform explosive coughs and then take full and deep breaths, filling lungs before expiring with moderate strength.

In order to access the collected breath condensate, the mouthpiece should first be removed from the EBC collection device for disposal.

The perforated label on the right hand side of the device should be broken and the parts should be folded apart from each other (appearing as a book pages open) allowing the EBC collection device to stand having the support of said two foldable flat parts. In case, fluid is reluctant to flow into the collection area (if there is an air trap), the collection area can be squeezed to push the air out and to allow the breath condensate to be pulled into the collection area when pressure is removed.

To access the exhaled breath condensate the EBC collection device is laid face down on a surface and the back part is opened. The small label covering the fluid collection area is peeled off and the required quantity of fluid is drawn out (e.g. by pipette) for tests.

The immunochromatographic testing is performed using single-use, disposable cartridges or strips which generate detectable coloured end products interpreted as positive or negative.

Accordingly, in the present invention the EBC instead of blood can be rapidly and efficiently analyzed for the presence of antigens and/or antibodies. Such analysis is less invasive and allows testing at home or in almost any other setting, not only in clinical settings.

The current methods are useful in any disease or clinical condition in which an ultrafiltrate (e.g., EBC, saliva or urine) can be assessed in conjunction genetic testing. For example, one skilled in the art can apply standard and available algorithms to identify appropriate sequences for PCR testing related to a sequence expressed in cancer or HIV infection.

The current methods are useful in any disease or clinical condition in which an ultrafiltrate (e.g. EBC, saliva or urine) can be assessed in conjunction serologic testing. For example, one skilled in the art can identify appropriate reporter antibodies for use in ICT, ELISA, or Western blots.

What is claimed is:

1. An exhaled breath condensate (EBC) collection device comprising two adjacent parts foldable together and apart from each other, a condensation zone to condense exhaled breath from a subject, the condensation zone being a third part provided between the two adjacent parts and connectable to cooling means arranged in each of the two parts being in cooling relationship with the condensation zone.

2. The device of claim 1, wherein the two adjacent parts and the condensation zone are essentially flat.

3. The device of claim 1, wherein the cooling means are freezer packs or ice packs.

4. The device of claim 1, further comprising an inlet port enabling the subject to breathe into the condensation zone through a detachable mouth-piece.

5. The device of claim 1, wherein the condensation zone comprises an exhaled breath condensate collection area in a bottom area of the condensation zone, enabling the generated breath condensate flow down to the collected area for subsequent collection for performing tests.

6. The device of claim 1, further comprising a perforated label connecting the two adjacent parts in a folded-together state.

7. The device of claim 1, further comprising a breathing indicator that is indicative of a rate of exhaled breath.

8. A method for detecting antigens or antibodies to the antigens, the method comprising:
collecting exhaled breath condensate (EBC) from a subject using an exhaled breath condensate collection device comprising two adjacent parts foldable together and apart from each other, a condensation zone to condense exhaled breath from a subject, the condensation zone being a third part provided between the two adjacent parts and connectable to cooling means arranged in each of the two parts being in cooling relationship with the condensation zone,
performing immunochromatographic testing (ICT) of the collected exhaled breath condensate, and
interpreting the immunochromatographic testing results as follows:
if the immunochromatographic testing results are positive, the antigens or antibodies to the antigens are detected, or
if the immunochromatographic testing results are negative, the antigens or antibodies to the antigens are not detected.

9. The method of claim 1, wherein the antigens are proteins.

10. The method of claim 1, wherein the method includes using a smartphone-based camera and/or app to perform quantitative assessments.

11. The method of claim 1, wherein the collecting of the EBC from the subject includes respiring by the subject through the EBC collection device at least for 5 minutes.
